(19) United States
(12) Patent Application Publication
(10) Pub. No.: US 2013/0131574 A1
(43) Pub. Date: May 23, 2013

(54) DIALYSIS TREATMENT MONITORING

(71) Applicants: Daniel L. Cosentino, Excelsior, MN (US); Laura M. Rudd, Mayer, MN (US); Brian A. Golden, Eden Prairie, MN (US); Kristin N. Parrott, Jordan, MN (US); Christopher T. Abrahamson, Bloomington, MN (US); Louis C. Cosentino, Juno Beach, FL (US)

(72) Inventors: Daniel L. Cosentino, Excelsior, MN (US); Laura M. Rudd, Mayer, MN (US); Brian A. Golden, Eden Prairie, MN (US); Kristin N. Parrott, Jordan, MN (US); Christopher T. Abrahamson, Bloomington, MN (US); Louis C. Cosentino, Juno Beach, FL (US)

(21) Appl. No.: 13/469,760

(22) Filed: May 11, 2012

Related U.S. Application Data
(60) Provisional application No. 61/484,983, filed on May 11, 2011.

(51) Int. Cl. A61M 1/30; A61M 1/28

(52) U.S. Cl. CPC A61M 1/30; A61M 1/28
USPC 604/6.07; 604/6.09; 604/28

(57) ABSTRACT

This disclosure describes dialysis treatment monitoring systems and methods for operating dialysis treatment monitoring systems. The disclosure describes novel systems and methods for collecting and processing dialysis treatment information from a patient.

### CROSS REFERENCE TO RELATED APPLICATIONS

The present application claims the benefit of U.S. Provisional Patent Application No. 61/484,983, filed on May 11, 2011, the disclosure of which is hereby incorporated by reference in its entirety.

### INTRODUCTION

The human body consists of approximately 60% water and maintaining this level is important for survival. While it is unproblematic to provide the body with new water, disposal of surplus water is a major problem in renal patients. One task of the kidneys is to remove superfluous fluid from the blood, such as water, urea and other waste products. The resulting urine is transferred to the bladder and finally leaves the body during urination. The kidney's second task is to regulate, for example, balance of electrolytes and acid and base. Disorders may develop in most major body organs due to malfunctioning kidneys. One such syndrome is called uremia. If uremia remains untreated, it will lead to death. Uremia is treated by kidney transplantation or some form of blood cleaning, either extracorporeal (e.g., in form of hemodialysis), or intracorporeal (e.g., in form of peritoneal dialysis).

Irrespective of which type of blood cleaning treatment that is used, the treatment requires a substantial amount of time, such as multiple sessions per day. Thus, for patient comfort and quality of life, it is vital that the treatments can be completed in a manner that is as straightforward and flexible as possible. To that end, various home dialysis solutions have been developed. These include home hemodialysis machines, continuous ambulatory peritoneal dialysis (CAPD) systems, and continuous cycler peritoneal dialysis (CCPD) systems.

Typically, home dialysis patients conduct their treatments at home as scheduled and then have a monthly visit to their Nephrologist and Home Dialysis Nurse, or other caregiver/monitoring nurse to review their treatment sheets, draw monthly labs, and/or adjust the care plan for the patient that defines the patient's dialysis treatment. Many times, these patients have to document their pre and post treatment information in a log which they provide to the physician at this visit.

This disclosure describes dialysis treatment monitoring systems, and methods for operating dialysis treatment monitoring systems. The disclosure describes novel systems and methods for collecting and processing dialysis treatment information from a patient.

In part, this disclosure describes a method for operating a dialysis treatment monitoring system. The method includes:

a) providing a patient monitoring system comprising a processor;

b) collecting dialysis treatment information relating to dialysis treatment of a patient using the processor, where collecting includes:
i) querying the patient to input dialysis treatment information using the processor, wherein the dialysis treatment information comprises medication information relating to one or more medications taken by the patient before, during or after the dialysis treatment; and
ii) receiving the dialysis treatment information from the patient through an input device using the processor;

c) transmitting the dialysis treatment information to a remote monitoring station

d) retrieving a patient log from a remote data store;

e) updating the patient log responsive to collecting dialysis treatment information or transmitting the dialysis treatment information; and

f) adjusting a care plan of the patient based on the updated patient log.

Yet another aspect of this disclosure describes a dialysis treatment monitoring system. The system includes an output device configured to present queries to a patient, wherein the queries are related to dialysis treatment of the patient; an input device configured to receive patient responses to the queries; a processor configured to collect and transmit the patient responses; and a remote processing location configured to receive the patient responses and determine whether an alert should be sent to a healthcare professional based on the patient responses.

Yet another aspect of this disclosure describes a dialysis treatment monitoring system. The system includes at least one communication device configured to connect a patient to an interactive voice recognition system, wherein the interactive voice recognition system collects dialysis treatment information from the patient through a series of queries and responses. The dialysis treatment information is transmitted to a remote processing unit. Also, the remote processing unit processes the dialysis treatment information and determines whether an alert should be sent to a healthcare professional.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF FIGURES

The following drawing figures, which form a part of this application, are illustrative of embodiments, systems and methods described below and are not meant to limit the scope of the invention in any manner, which scope shall be based on the claims appended hereto.

FIG. 1 is a flow chart of one example of a method for operating a dialysis treatment monitoring system;

FIG. 2 is a schematic diagram of one example of a dialysis treatment monitoring system; and

FIG. 3 is a schematic diagram illustrating one example of a dialysis treatment monitoring system.

### DETAILED DESCRIPTION

Although the methods, apparatuses, and systems introduced above and discussed in detail below may be useful in a variety of healthcare environments, the present disclosure will discuss the implementation of these techniques for use in remotely monitoring an ambulatory patient. The technology described in the context of a home-healthcare environment could be adapted for use in other healthcare environments that would benefit from remote-monitoring of a patient condition.

Embodiments of the dialysis treatment monitoring system assist in home dialysis treatment monitoring, assessment, and intervention. Some embodiments provide the patient's care team with “real-time” data capture. The provider can use this data to recognize and intervene on situations (such as a narrowing in the dialysis access) based on the data that is sent received after each treatment is complete. This data can also be used to monitor and adjust treatment medications in a much timelier manner than current practices and systems. This may result in the avoidance of potential hospitalizations by providing early intervention.

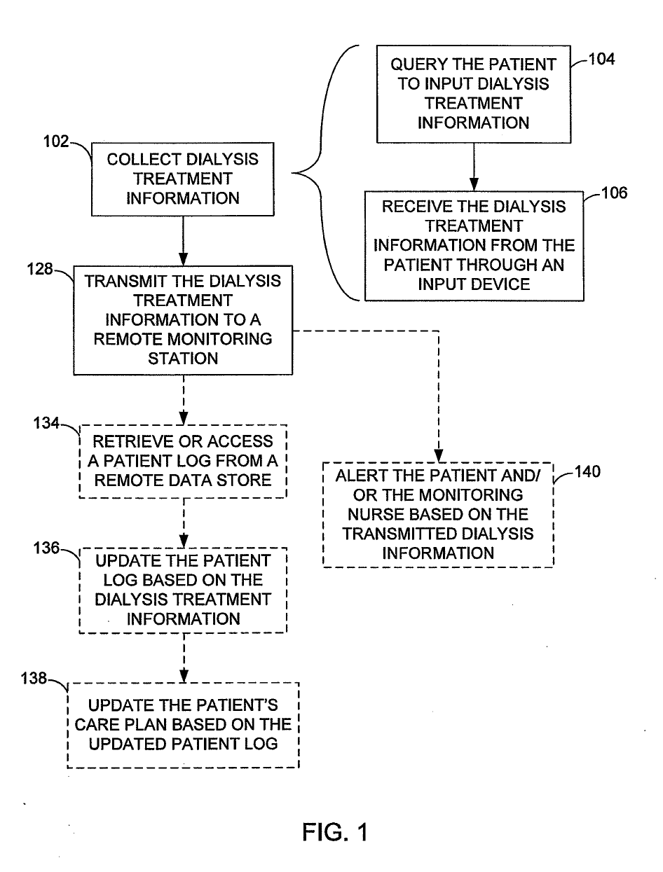

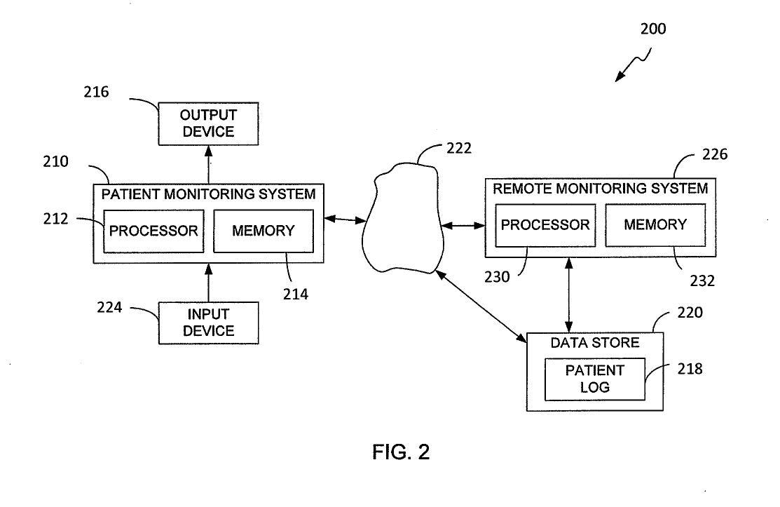

FIG. 1 is a flowchart illustrating a method 100 in accordance with embodiments of the dialysis treatment monitoring system. FIG. 2 is a simplified diagram of a dialysis treatment monitoring system 200 in accordance with several embodiments of the dialysis treatment monitoring system. In one embodiment, the dialysis treatment monitoring system 200 can be used to perform the method 200. For purposes of explanation, the method 100 will be explained as implemented by system 200. However, it is understood that the method 100 may be implemented by a variety of different systems and apparatuses.

Now referring to FIG. 1, dialysis treatment information is collected and received at 102. The dialysis treatment information generally relates to a dialysis treatment of a patient. Exemplary dialysis treatment information may include, for example, medication information, fluid information including information relating to the dialysate used in a dialysis treatment, symptom information relating to potential symptoms the patient may be experiencing in relation to the dialysis treatment, information relating to a home dialysis unit (e.g., hemodialysis machine or peritoneal dialysis cycler), and/or other information relating to the dialysis treatment of the patient. Embodiments of the information relating to the home dialysis unit may include, for example, the machine temperature, the dialysate flow rate, the blood flow rate, the length of the treatment, the amount of fluid given during the treatment, the amount of fluid recovered during the treatment, the sodium level or sodium given during the treatment, the total saline used during the treatment, Kt/V, and other information. The dialysis treatment information may also include physiological parameters of the patient, such as blood pressure (e.g., before, after and during the treatment), temperature, weight (before and after the dialysis treatment), qualitative or quantitative urine analysis information, and other information. Other exemplary embodiments of this dialysis treatment information are described below in greater detail.

In one embodiment of the method step 102, the patient is queried to input the dialysis treatment information, as indicated at 104. Next, some or all of the dialysis treatment information is received from the patient through an input device, as indicated at 106. The particular manner in which method steps 104 and 106 are carried out depends on the dialysis treatment monitoring system 200 that is being used.

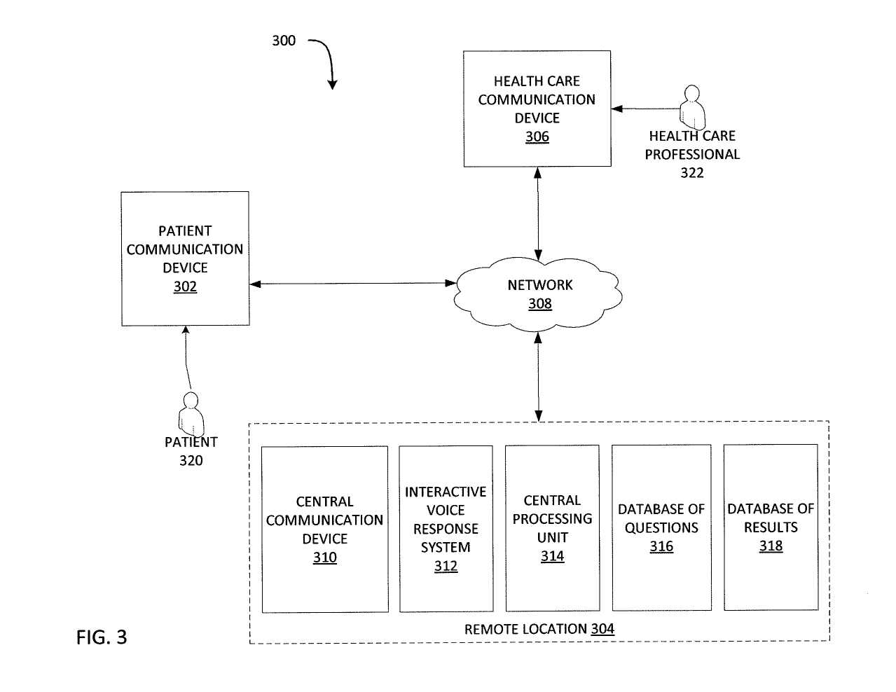

For example, the steps 104 and 106 may be carried out utilizing an interactive voice response (IVR) system. The IVR system allows patients to call into the system using their telephone without any additional equipment. However, in one embodiment, the IVR system is supplemented with a patient monitoring system in accordance with one or more of the embodiments described below. The IVR system prompts the patient with queries (step 104) and allows the patient to respond with a voice response and/or a touch-tone keypad response to provide the requested dialysis treatment information (step 106). Responses may be made available to the caregiver immediately during the call or after the patient's phone call has ended. In one embodiment, the IVR system may be designed to call patients at predetermined times in response to an alert, which may be generated by the system in accordance with embodiments described below. More information about the IVR system is discussed below with reference to FIG. 3.

A patient monitoring system 210 is used to carry out the method steps 104 and 106 in an alternate embodiment. For example, the patient monitoring system 210 includes one or more processors, which are represented by processor 212. In accordance with another embodiment, the system 210 includes a memory 214 that is accessible by the processor 212. The memory 214 may be local to the system 210, as shown in FIG. 2, or located remotely from the processor 212. Embodiments of the dialysis treatment monitoring system include instructions stored in the memory 214 that may be executed by the processor 212 to perform method steps and other functions described herein including receiving data, processing data, communicating data, displaying data and other functions. Embodiments of the system 210 also include communication circuitry, which is used by the processor 212 to receive and transmit data in accordance with conventional wired and wireless communication protocols.

The system 210 either includes an output device 216, or is otherwise configured to communicate with an output device 216 in accordance with conventional data communication protocols. The output device 216 is used to present the queries to the patient in accordance with step 104 of the method. Exemplary embodiments of the output device 216 includes a display device, a speaker, a telephone, a computer, a personal digital assistant (PDA) and/or other output device that may be used to communicate queries to the patient. Thus, for instance, when the output device 216 is in the form of a display device, queries are presented on the display device to direct the patient to input dialysis treatment information to the system 210.

In one embodiment, the patient monitoring system 210 may include a patient input device and a patient communication device. The patient communication device may work in conjunction with the patient input device to collect dialysis treatment information from the patient. The communication device may then transmit this data to the remote location. In alternate embodiments, the system 210 includes a purpose-built device which acts as both the communication device and the input device by providing a patient communication device with a user interface. In yet further embodiments, the patient may utilize a communication device having a browser to access a webpage which interacts with the patient. The patient may interact with the webpage to communicate dialysis treatment information and act as an input device. The webpage may transmit the information over a network to the remote location for processing. Alternatively, the patient communication device may include software, which when run, is configured to interact with the patient to collect dialysis treatment information and then transmit this information to the remote location. In the above embodiments, the patient communication device may be any computing device with communications capabilities such as a desktop computer, laptop computer, smart phone, tablet computer, or the like. For example, the patient communication device may be an iPhone, iPad, iPod, Blackberry, or the like. The queries of step 104 may be general queries that are used to extract dialysis treatment information from a general patient undergoing dialysis treatment. Such queries may be organized in a hierarchical manner such that the queries step through different pathways within the hierarchy of questions based on the responses provided by the patient. Such queries may be generated or selected for presentation to the patient through the output device 216 using the processor 212. In one embodiment, the queries are presented in a multiple choice format. In accordance with another embodiment, the queries are presented to the patient in a manner that requires the patient to input a full response to the query, in the form or a sentence, a word, or the like. Embodiments of the queries include other forms as well. In an embodiment, the questions are stored in a table on the memory 214. The questions may be precached in the memory 214 and periodically updated as needed so that such questions do not need to be downloaded each time the system is used. In an alternative embodiment, the questions may be retrieved from the remote monitoring at the start of each session or during a session in real time as needed.

The queries presented in step 104 may also be customized to the specific patient being addressed by the queries. These patient-specific queries are generated in response to patient information that has been collected by the system 200. In one embodiment, this patient information is stored in a patient log 218 stored in the memory 214 or a data store 220 located remotely from the patient monitoring system 210, which is accessible through a network 222 or other conventional technique. In one embodiment, the query log 218 indicates, for example, the type of dialysis treatment the patient is undergoing (i.e., hemodialysis, CAPD, CCPD), dialysis treatment-related medication prescriptions for the patient, and other information that is unique to the patient.

Additionally, the query log 218 may include historical information relating to dialysis treatments the patient has undergone. Exemplary embodiments of this historical information include information on specific dialysis treatments, medications taken by the patient, and other historical dialysis treatment information. This historical data may also be used to narrow the queries presented to the patient. For instance, rather than querying the patient to identify one or more medications the patient is taking relating to the dialysis treatment, the patient monitoring system 210 may instead directly ask the patient if he or she has taken a specific medication that is identified in the patient log 218.

In one embodiment, system 200 includes an input device 224 that is either included in the patient monitoring system 210 or configured to communicate with the patient monitoring system 210 using a conventional communication technique. The input device 224 is generally used by the patient to input the dialysis treatment information to the patient monitoring system 210 and carry out the method step 106. The input device 224 may take on many different forms and a patient monitoring device may utilize multiple, different input and output devices. Exemplary embodiments of the input device 224 include, a blood pressure monitor, a telephone (such as for use within 1\TR system), a dialysis unit (e.g., a hemodialysis machine or a peritoneal dialysis cycler), a scale, a computer, a tablet computer, a smart phone, a microphone, a webpage interface, a keyboard, a keypad and/or other suitable input device.

The input device 224 acquires the desired dialysis treatment information and provides the information to the patient monitoring system 210. For instance, when the input device 224 is in the form of a scale, the patient may be queried (step 104) to stand on the scale, or place an item (e.g., bag of dialysate) on the scale. The weight of the item can then be delivered directly to the patient monitoring system 210.

Following the collection of the dialysis treatment information (step 102) the dialysis treatment information is transmitted to a remote monitoring system 226, as indicated at step 128. Embodiments of the remote monitoring system 226 include a computer, such as a work station of a caregiver, a server, which may act as a host for network clients to access dialysis treatment information of the patient, a mainframe, one or more databases, a distributing computing environment, and/or other suitable computing network. One embodiment of the remote monitoring system 226 includes one or more processors, which are represented by processor 230. In accordance with another embodiment, the system 226 includes memory 232. The memory 232 may be located locally to the system 226 or remotely from the system 226. The processor 230 is generally configured to execute instructions stored in the memory 232 and perform method steps and functions in accordance with embodiments described herein. The system 226 also includes conventional communication circuitry for data communications using the processor 230. Thus, the remote monitoring system 226 is configured to receive the dialysis treatment information transmitted in step 128 from the patient monitoring system 210 using the processor 230. The system 226 may also store the dialysis treatment information in the memory 232 or the remote data store 220 in the patient log 218 corresponding to the patient.

Additional embodiments of the method and optional steps are illustrated in FIG. 1 in phantom. In one embodiment, the patient monitoring system 210 retrieves a patient log 218 corresponding to the patient from a remote data store 220 at step 134. This may be accomplished by the system 210 in accordance with any suitable technique. For instance, the system 210 may directly access the patient log 218 from the remote data store 220 using processor 212.

The patient monitoring system 210 may also request the patient log 218 from the remote monitoring system 226. Thus, in accordance with an alternative embodiment, the remote monitoring system 226 retrieves the patient log 218 from the data store 220 or the memory 232 at step 134.

Next, at 136, the patient log 218 is updated based on the dialysis treatment information transmitted at step 128. In one embodiment, after the remote monitoring system 226 receives the dialysis treatment information, the remote monitoring system 226 modifies the patient log 218 to update the information contained in the patient log 218 based on the dialysis treatment information. This update of the patient log 218 may involve updating medication information in the log 218 (e.g., medications taken by the patient before, during and/or after the most recent dialysis treatment), updating information regarding the most recent dialysis process performed on the patient (date of the treatment, duration of the treatment, amount of dialysate used during the treatment, and/or other dialysis treatment information), updating symptom information, updating information relating to the dialysate fluid used during the most recent dialysis treatment, and/or updating or adding other information relating to the transmitted dialysis treatment information.

The patient log 218 is generally used to document that the patient's pre and/or post-dialysis treatment information in a log that they provide to the physician, Nephrologist, home dialysis nurse, or other caregiver during a regular visit (e.g., monthly). Thus, the patient's home dialysis information for the patient's daily treatments may be communicated and logged by the system 200. This provides the patient's care team with “real-time” data capture. Another benefit to this real time data provided in the patient log 218 is to monitor and adjust treatment medication or other parameters of the dialysis treatment of the patient in a much timelier manner. This approach to monitoring a patient's dialysis treatment can prevent hospitalizations of the patient by avoiding the delay inherent in conventional dialysis treatment monitoring practices.

At step 138, the patient's care plan is updated based on the updated patient log 218. The care plan may be contained within the patient log 218 or may be stored as a separate document in the data store 220 or the memory 232. The care plan generally defines the patient's dialysis treatment including various parameters of the dialysis treatment. In one embodiment of the method step 138, one or more of the parameters of the patient's dialysis treatment is adjusted based on the update to the patient log 218. This adjustment may be responsive to the aggregation of various dialysis treatment information, such as medication information, symptom information, fluid information, dialysis unit information, and other information contained in the patient log 218. This adjustment to the patient's dialysis treatment care plan may be made automated by the system 200 through a communication with the patient using the patient monitoring system 210 or IVR system. In accordance with another embodiment, the care provider utilizes the updated patient log 218 to determine whether an adjustment to the care plan for the patient is necessary. If an adjustment is necessary, the care provider may communicate the adjustment using the system 200, or during the next regularly scheduled meeting with the patient.

The patient and/or the monitoring nurse (i.e., the caregiver, healthcare professional, etc.) may be alerted based on the transmitted dialysis information (step 128), as indicated at 140. In one embodiment, the patient and/or caregiver is alerted by the remote monitoring system 226, the patient monitoring system 210, or the IVR system. Such an alert may be in the form of an automated phone call, an email, a text message, a page, or other notification.

In some embodiments, step 140 involves an analysis of the dialysis treatment information transmitted by the patient. This analysis involves comparing the dialysis treatment information transmitted in step 128 to information contained in the patient log 218. In one embodiment, the alert is generated responsive to one or more of the dialysis treatment parameters falling outside predefined boundaries. For instance, the analysis may involve a comparison of one or more of the parameters contained in the dialysis treatment information to determine whether the degree of change of the parameter from a prior dialysis treatment to the current dialysis treatment has fallen outside a predefined boundary. The boundaries for a given parameter may be defined, for example, by a threshold level that is exceeded by a value corresponding to the dialysis treatment parameter, a threshold level that the value of the dialysis treatment parameter drops below, or a threshold change in a value of a dialysis treatment parameter. In one embodiment, this analysis is performed automatically by the remote monitoring system 226 or the patient monitoring system 210 using the corresponding processor.

Instructions or information may be provided to the patient based on the dialysis treatment information collected in step 102. This may be accomplished by the patient monitoring system 210 using the processor 212 or the remote monitoring system 226 using the processor 230. In one embodiment, the instructions are provided to the patient using the output device 216. Thus, the instructions may be presented on a display device, up through a speaker, communicated to the patient through a telephone communication, or presented to the patient in another form using the output device 216.

Exemplary instructions may include instructions or information on using the patient monitoring system 210 including entering dialysis treatment information or performing other tasks using the system 210. The information may also relate to the patient's dialysis treatment such as, for example, instructions regarding how the dialysis treatment should be conducted by the patient and other information. The instructions may also include instructions regarding the one or more medications taken by the patient, such as, for example, how the medication should be taken, the dosage of the medication to be taken, the frequency at which the medication should be taken, and other instructions. In one embodiment, the instructions or information include educational information, such as information relating to the dialysis treatment process, information regarding how to improve ones' health, and other information. Other exemplary instructions include instructions to change a medication dosage, instructions to change a frequency of which a medication is taken, instructions to change a medication, instructions to contact a caregiver, instructions to provide additional dialysis treatment information, and other instructions.

### Medication Information

As mentioned above, one embodiment of the dialysis treatment information includes medication information relating to one or more medications taken by the patient before, during and/or after a current dialysis treatment. Such medications may include, for example, Epogen, Heparin, an iron supplement, a calcium supplement, a vitamin, a mineral, or other medication used in connection with dialysis treatments. In one embodiment, the medication information includes information regarding the dialysate used in the dialysis treatment, such as an identification of the one or more medications added to the dialysate.

One embodiment of the query step 104 comprises presenting a question to the patient relating to whether a medication was taken, what medication was taken, when the medication was taken, how often the medication is taken, how much of the medication was taken, and/or how the medication was taken. Such queries may be presented to the patient using numerous techniques depending on the output device 216, such as those described above.

The method may involve accessing medication prescription information for the patient. In one embodiment, this is performed by the patient monitoring system 210 using the processor 212. In one embodiment, this is performed by the remote monitoring system 226 using the processor 230. In one embodiment, the medication prescription information is contained within the patient log 218 stored in the data store 220. In accordance with another embodiment, the medication prescription information is stored separately from the patient log 218. Exemplary embodiments of the medication prescription information include an identification of a prescribed medication, a prescribed dosage of the prescribed medication, a prescribed frequency for taking the prescribed medication at the prescribed dosage, or other information relating to a medication prescription.

The query step 104 involves presenting a patient-specific query that is based on the medication prescription information. For instance, the query presented in step 104 may ask “Did you take medication X during your last dialysis treatment?”, “Did you take X units of medication Y?” etc. In other words, in one embodiment the hierarchical set of queries, which may be contained in the memory 214, the memory 232 or the data store 220, from which the query is generated in step 104, may be filtered based on the medication prescription information for the patient. Additionally, query pathways may also be filtered if they are not applicable to the particular patient.

In one embodiment, the method involves updating the patient log 218 stored in the remote data store 220 responsive to the medication information transmitted in step 128. This embodiment is generally accomplished in accordance with method steps 134 and 136 described above. In one embodiment, the updating of the patient log 218 includes updating a care plan (step 138) for the patient responsive to or based on the medication information provided in step 128. In one embodiment, this update to the care plan includes adjusting one of the medications being used by the patient or adding a new medication to the care plan for the patient. In other exemplary embodiments, the adjusting of one of the medications taken by the patient includes adjusting a dosage of the medication, a frequency at which the medication is taken, the time when the medication is taken, or other adjustment.

The method may provide an alert (step 140) to the patient or the caregiver when the medication information provided by the patient does not match medication prescription information stored in the data store 220 or other location. As discussed above, one embodiment of the system includes medication prescription information for the patient stored in the remote data store 220 either in the patient log 218 or a separate medication log for the patient. In one embodiment of the alerting step 140, the medication prescription information is retrieved or accessed from the data store 220. The medication prescription information for the patient is then compared to the medication information transmitted in step 128.

This analysis is automated and performed by the remote monitoring system 226 used in the processor 230 or the patient monitoring system 210 using the processor 212. Alternatively, this analysis of the medication prescription information and the medication information contained in the dialysis treatment information may be performed by the caregiver. In one embodiment, the patient and/or the monitoring nurse or caregiver is alerted responsive to a difference between the prescription information and the medication information. As a result, the patient and/or the caregiver may be alerted to a discrepancy between the medication information provided by the patient and the medication prescription information. For example, if a patient typically did not add any heparin to their dialysate fluid, but now the medication information entered by the patient responsive to the queries (104) indicates that the patient added heparin to the fluid, an alert may be generated (104) to notify the caregiver of the change. Patients are taught to add the heparin to the dialysate bag if they see fibrin in their waste bags, which can be a sign peritonitis and possible catheter blockage. The caregiver may then intervene to see why the patient made the adjustment to the prescribed dialysis treatment.

The medication information is analyzed by the remote monitoring system 226 using the processor 230 or the patient monitoring system 210 using the processor 212 to determine whether a parameter of the medication information falls outside a predefined boundary as discussed above. An alert (step 140) can then be provided to the patient and/or the caregiver to provide notice of the out of bounds finding by the system 226 or the system 210. In accordance with one embodiment, the alerting of the patient and/or the caregiver may be followed by an adjustment to the care plan for the patient, such as an adjustment to the dosage level of one of the medications, a frequency at which one of the medications is taken, or the adjustment of another parameter relating to the medications taken by the patient. This adjustment may be automatically suggested by the system 226 or the system 210, or the adjustment may be made by the caregiver, as discussed above. Alternatively, this update to the patient's care plan may be made in accordance with step 138 of the method.

In accordance with another embodiment of the method, the patient log 218 is retrieved or accessed from the remote data store 220. This may be accomplished by the patient monitoring system 210 using the processor 212 or the remote monitoring system 226 using the processor 230. The dialysis treatment information is then compared to information contained in the patient log 218. An alert is then provided to the patient, the caregiver, or both, responsive to the comparison of the dialysis treatment information to the patient log.

### Fluid Information

As mentioned above, additional embodiments of the dialysis treatment monitoring system relate to dialysis treatment information in the form of fluid information relating to the dialysate used in the dialysis treatment of the patient and the waste fluid or dialysate effluent recovered during the dialysis treatment. Exemplary embodiments of the fluid information include information identifying the dialysate used in the dialysis treatment, a volume or weight of administered dialysate, a volume or weight of the dialysis effluent recovered during the dialysis treatment, an identification of a medication or additive (e.g., dextrose, heparin, glucose, etc.) within the dialysate, a concentration of the medication or additive within the dialysate, a dosage of the medication or additive within the dialysate, and other information relating to the dialysate. Such fluid information related to urine may also be obtained and utilized.

The patient may be queried (step 104) to provide a weight or volume of the dialysate before the current dialysis treatment begins. In one embodiment, the patient utilizes an input device 224 in the form of a scale to measure the weight of the dialysate, which is typically provided in a bag. In accordance with another embodiment, the patient is queried (step 104) to also provide the weight or volume of the dialysate effluent recovered during the dialysis treatment. In one embodiment, the system 210 or patient utilizes the input device 224 in the form of the scale to obtain the weight of the dialysis effluent.

Alternatively, the patient is queried (step 104) to provide his or her weight before and after the dialysis treatment. In one embodiment, this is accomplished using an input device 224 in the form of a scale. The monitoring system 210 using the processor 212 or the remote monitoring system 226 using the processor 230 can use this weight information to determine the amount of fluid administered to the patient and the amount of fluid discharged from the patient during the dialysis treatment.

The fluid information may be used to update the patient log 218 (steps 134 and 136). Additionally, the fluid information may be used to update the patient's care plan (step 138) either automatically by the system 210 or 226, or by the caregiver. Exemplary adjustments to the care plan include instructions to change the dialysate, instructions to change a volume or weight of the dialysate, instructions to change a medication within the dialysate, instructions to change a concentration of a medication within the dialysate, instructions to change a dosage of a medication within the dialysate, and/or other instructions relating to the dialysate.

Additionally, the fluid information provided by the patient (step 102) may be used to trigger an alert (step 140). The alert may be triggered, for example, responsive to a comparison of the patient provided fluid information and the patient log, as discussed above.

### Symptom Information

In one embodiment, the dialysis treatment information includes symptom information that may relate to an access of the patient through which the dialysate is fed into the patient, such as a catheter. Exemplary embodiments of this symptom or access information relates to information that may be used to determine if the access is infected, blocked, or has another problem that may require attention. According to this embodiment, the query step 104 may present queries to the patient relating various symptoms involving the access, such as whether the area surrounding the access is swollen, red, hot, sore or has another associated symptom that may indicate a problem with the access. Exemplary symptom information provided by the patient (step 106) responsive to the queries include an assessment of an amount of swelling around a dialysis treatment access to the patient, an assessment of a level of redness around the dialysis treatment access, a soreness of the area around the dialysis treatment access, a length of a catheter, and other information.

Other queries include determining whether there is a change in the flow rate through the access. In one embodiment, the dialysis treatment information includes a dialysis flow rate that may be obtained from the input device 224 in the form of a dialysis unit (hemodialysis machine, peritoneal dialysis machine) or the flow rate information may be input manually by the patient. The flow rate may be measured by the dialysis unit or determined based on the duration of the dialysis treatment. In one embodiment, the patient monitoring system 210 or the remote monitoring system 226 compares the dialysate flow rate of the current dialysis treatment with corresponding information within the patient log 218 to determine whether a problem may exist. In one embodiment, the comparison involves analyzing the information for a change in the dialysate flow rate or a flow rate value that falls outside predefined boundaries. If the analysis finds that the dialysate flow rate provided by the patient (step 106) or otherwise collected by the system 200 (step 102) falls outside a predefined boundary, an alert (step 140) may be sent to the patient and/or the caregiver for the patient. In one embodiment, the caregiver contacts the patient in response to the alert.

Other exemplary embodiments of the symptom information relate to potential symptoms the patient may be experiencing in relation to the dialysis treatment. Examples of this symptom information include a color of the dialysate effluent, a cloudiness level of the dialysate effluent, a temperature of the patient, a blood pressure of a patient, a weight of the patient before the dialysis treatment, a weight of the patient after the dialysis treatment, or other symptom information. This information is contained in the dialysis treatment information collected in step 102 of the method, and can be used to update the patient log 218 (steps 134 and 136), update a care plan (step 138) and/or trigger an alert (step 140), in accordance with the embodiments described above.

The dialysis treatment information collected in step 102 of the method may include a photo acquired using an input device 224 in the form of a digital camera or a photo retrieved from a memory (e.g., flash memory, local memory, remote memory, etc.). The digital photo is then provided or received (step 106) by the patient monitoring system 210. The photo may be stored in the memory 214, or transmitted in step 128 of the method to the remote data store 220 or other memory location.

In some embodiments, the system instructs the patient to (step 104) to acquire a particular photo using the input device 224 or otherwise provide the photo. In one exemplary embodiment, the query in step 104 requests that the patient take a photo of the dialysate effluent. One purpose for capturing a photo of the dialysate effluent is to determine whether a patient may have an infection or other condition that is indicated by the color or cloudiness of the dialysate effluent. In accordance with another exemplary embodiment, the query in step 104 requests that the patient take a photo of the area around the dialysis treatment access. Such a photo may indicate swelling or redness around the dialysis treatment access which may indicate an infection at the access, a blockage through the access, or other condition that may be accessed remotely by the caregiver based on the photo. The photo may be analyzed by the caregiver who may contact the patient if the photo indicates a potential problem.

The patient monitoring system 210, using the processor 212, may provide instructions to the patient responsive to the transmission of the symptom information in step 128 of the method. These instructions may include instructions for treating a condition of the patient that is indicated by the symptom information. In one embodiment, these instructions are automated by the patient monitoring system 210 or the remote monitoring system 226. In accordance with another embodiment, the instructions are provided directly to the patient by a caregiver. In one embodiment, these instructions are provided responsive to the generation of an alert by the patient monitoring system 210 or the remote monitoring system 226 (step 140) based on the system information provided by the patient. This alert may be generated responsive to a comparison of the symptom information to information contained in the patient log 218, as described above with regard to other embodiments of the dialysis treatment monitoring system.

Embodiments of the dialysis treatment monitoring system also include the application of the above described embodiments to a population of patients undergoing home dialysis treatments. One advantage of the system and methods of the dialysis treatment monitoring system is that large populations of patients undergoing dialysis treatment may be monitored by relatively few caregivers.

### Accessing the Dialysis Treatment Monitoring System Through Interactive Voice Response (“IVR”) System

Referring now to FIG. 3 a schematic diagram illustrating one example of a dialysis treatment monitoring system 300 is shown. The dialysis treatment monitoring system 300 includes a general-purpose patient communication device 302, remote processing system 304, a health care communication device 306, and a network 308. The remote processing system 304 includes a central communication device 310, an interactive voice response (“IVR”) system 312, a central processing unit 314, a database of questions 316, and a database of results 318. The general-purpose patient communication device 302 can be accessed and operated by a patient 320. The health care communication device 306 can be accessed and operated by a health care professional 322.

The central communication device 310 is located at the remote processing system 304. The remote processing system 304 is one embodiment of the remote monitoring system 226 of FIG. 2. Examples of the central communication device 320 may include a telephone, a cellular telephone, a pager, a tablet computer, a computer, or other wireless or wired communication devices. When the patient 320 initiates a communication session with the central communication device 310, the central communication device 310 communicates with the IVR system 312 which interacts with the patient 320 through a series of queries and/or responses based on answers provided by the patient 320, as discussed above.

The IVR system 312 serves as a front end interface through which a caller, such as the patient 320, can access the patient health monitoring system 300. The IVR system 312 acts as other interactive voice response systems that are known in the art. Specifically, the IVR system 312 provides prompts to the patient 320 and receives touch tone and/or spoken responses from the patient 320 in response to the prompts. The IVR system 312 communicates with the central processing unit 314 to determine which queries to present to the patient 320. The central processing unit 314 accesses the database of questions 316 and transmits the questions to the IVR system 312. Upon receiving a response from the patient 320, the IVR system 312 once again communicates with the central processing unit 314, which stores and updates the answers in the database of results 318. Through this series of prompt/response interaction, the IVR system 312 collects sufficient information about the dialysis treatment of the patient 320 to determine a health status of the patient 320.

The central processing unit 314 accesses the database of questions 316 based on the information acquired by the IVR system 312. The database of questions is programmed with several question hierarchies having varying numbers of sets of questions associated with each question hierarchy.

The health care communication device 306 may be located at a health care professional's office, health care oversight location, or any other location that is easily accessible to the health care professional 322. The health care professional 322 can access the information gained through the IVR system 312, which is stored in the database of results 318. In some embodiments, the health care professional 322 may be located at the remote processing system 304 and therefore has direct access to the central processing unit 314. In other embodiments, the health care professional 322 may be located at a different location than the remote processing system 304 and thus may access the central processing unit 314 through the health care communication device 306 by way of the network 308.

In use, the patient 320 may begin interaction with the IVR system 312 through various ways. For example, in one embodiment, the patient 320 may initiate interaction with the IVR system 312 by calling a specific telephone number which connects the patient 320 to the IVR system 312 by way of the network 308. In this example, the network 308 may be a switched telephone network. In another embodiment, the health care professional 322 or some third party may initiate interaction between the patient 320 and the IVR system 312 by prompting the central communication device 310 to call the patient 320. In this example, the patient 320 may choose to interact with the IVR system 312 by answering the call upon receiving it.

In some embodiments, upon initiating interaction with the IVR system 312, the patient 320 may be prompted to input an identification code so that the IVR system 312 can identify the patient 320. The identification code may also be used to ensure that the person engaging with the IVR system 312 is authorized to do so. The patient 320 may input this information either through touch tone (utilizing a dial pad) and/or spoken responses. In some embodiments, the identification code may include a name, unique identification number such as a social security number, or other distinctive number or feature associated with the patient 320 to help the IVR system 312 identify the patient 320. In other embodiments, the IVR system 312 may not prompt the patient 320 for an identification code, but may instead, recognize the telephone number from which the patient 320 is calling, and utilize this telephone number as an identification code.

After identifying the patient 320, the IVR system 312 begins prompting the patient with health-related questions retrieved from the database of questions 318. The patient 320 can respond to the questions either through touch tone and/or spoken responses. The IVR system 312 then transmits the responses from the patient 320 to the central processing unit 314 which updates the database of results 318 with the patient responses. The central processing unit 314 then utilizes the responses to determine what to present next to the patient 320. For example, in some embodiments, the IVR system 312 may prompt the patient 320 with further questions from the same or different question hierarchies. In other embodiments, the IVR system 312 may present the patient 320 with feedback in response to answers which may include comments on how to improve the patient's health, general statements regarding the patient's health status and/or dialysis treatment, teaching tips on how to maintain the patient's health, or the like. In yet other embodiments, the IVR system 312 may complete a question hierarchy and determine (via the central processing unit 314) that no further questions or feedback need to be presented to the patient 320 and therefore terminate the call. Thereafter, the remote system 304 may update the patient log and/or the patient care plan based upon the newly acquired dialysis treatment information.

In some embodiments, the remote processing system 304 may utilize data collected from a plurality of different monitoring devices to determine whether more information is needed. If more information is desired to properly determine the patient's health status, the remote processing system 304 may initiate a call to the patient through the IVR system 312 so that it may prompt queries to the patient relevant to the information that the system 304 desires.

The central processing unit 314 may determine that the patient 320 is in need of professional medical assistance. In this case, the IVR system 314 may automatically utilize the central communication device 310 to initiate communication with the health care communication device 306 or send an alert to the health care profession 322 indicating that the patient 320 is in need of medical assistance because one or more of the patient's dialysis treatment parameters fall outside predefined boundaries. Alternatively, the IVR system 314 may prompt the patient 320 to call a health care professional at the patient's convenience.

Alternatively, the central processing unit 314 may determine that the patient 320 is in need of a follow-up consultation with the IVR system 312 within a certain time period after the present call. The central processing unit 314 may add the patient 320 to a prepared queue of callers that the IVR system 312 has been programmed to call automatically. Such a queue may be updated by a health care professional and/or the central processing unit 314. As such, the IVR system 312 will automatically initiate interaction with the patient 320 at a predetermined time in the future based on the patient's position in the queue.

In some embodiments, the patient communication device 302 may be a computing device with communications capabilities, such as a desktop computer, smart phone, tablet computer, or the like. For example, the patient 320 may interact with the remote location 304 by way of the patient computing device, wherein the patient computing device connects to a web server through the network 308. The patient 320 can utilize the computing device to open a web browser to initiate a communication session with the web server. In the example, the web server receives an initial page request, which is processed by the web server. A web page is then generated by the central processing unit 314. The page is transmitted to the web server which then sends the data over the network 308 to the patient computing device which displays the page to the patient via the web browser. At this time, the patient 320 may begin interaction with the web server, via the web page, through the web browser. The patient 320 may then begin responding to question hierarchies presented to the patient via the web browser, in a similar fashion as described above.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternative embodiments having fewer than or more than all of the features herein described are possible.

While various embodiments have been described for purposes of this disclosure, various changes and modifications may be made which are well within the scope of the present disclosure. Numerous other changes may be made which will readily suggest themselves to those skilled in the art and which are encompassed in the spirit of the disclosure and as defined in the appended claims

What is claimed is:

1. A method comprising:
providing a patient monitoring system comprising a processor;
collecting dialysis treatment information relating to dialysis treatment of a patient using the processor, wherein collecting comprises:
querying the patient to input dialysis treatment information using the processor, wherein the dialysis treatment information comprises medication information relating to one or more medications taken by the patient before, during or after the dialysis treatment; and
receiving the dialysis treatment information from the patient through an input device using the processor;
transmitting the dialysis treatment information to a remote monitoring station
retrieving a patient log from a remote data store;
updating the patient log responsive to collecting dialysis treatment information or transmitting the dialysis treatment information; and
adjusting a care plan of the patient based on the updated patient log.

2. The method of claim 1, wherein querying the patient to input dialysis treatment information using the processor comprises presenting a query to the patient on a display device and/or through an interactive voice response (IVR) system.

3. The method of claim 1, wherein the input device is selected from the group consisting of a dialysis unit, a scale, a computer, the monitoring system, a microphone, a web page interface, a keyboard, and/or other input device described herein.

4. The method of claim 1, wherein adjusting the patient's care plan based on the updated patient log is performed by a caregiver.

5. The method of claim 1, further comprising:
retrieving medication prescription information from a remote data store; comparing the medication prescription information to the medication information; and
alerting at least one of the patient and a monitoring nurse responsive to a difference between the prescription information and the medication information.

6. The method of claim 5, wherein:
the medication prescription information comprises a prescribed dosage of a prescribed medication;
the medication information comprises an administered dosage relating to the dosage of the prescribed medication taken by the patient;
comparing the medication prescription information to the medication information comprises comparing the prescribed dosage to the administered dosage; and
alerting at least one of the patient and a monitoring nurse comprises alerting one of the patient and a monitoring nurse responsive to a difference between the prescribed dosage and the administered dosage.

7. The method of claim 1, wherein the one or more medications are selected from the group consisting of Epogen, Heparin, an iron supplement, a calcium supplement, a vitamin, and a mineral.

8. The method of claim 1, wherein the medication information comprises information regarding a dialysate use in the dialysis treatment.

9. The method of claim 1, wherein querying the patient to input dialysis treatment information using the processor comprises presenting a question relating to whether a medication was taken, what medication was taken, when the medication was taken, how often the medication is taken, how much of the medication was taken, and/or how the medication was taken.

10. The method of claim 9, further comprising accessing the patient log from a remote data store using the processor, the patient log comprising medication prescription information selected from the group consisting of an identification of a prescribed medication, a prescribed dosage of the prescribed medication and a prescribed frequency for taking the prescribed medication at the prescribed dosage.

11. The method of claim 10, wherein querying the patient comprises presenting a query based on the medication prescription information.

12. The method of claim 1, further comprising reducing a remaining available dosage of one of the medications in the patient log responsive to the medication information.

13. The method of claim 5, wherein alerting at least one of a patient and monitoring nurse comprises instructing the patient to change at least one of a dosage level of one of the medications and a frequency at which one of the medications is taken, using the processor.

14. The method of claim 1, further comprising:
comparing the dialysis treatment information to information contained in the patient log; and
alerting at least one of the patient and a monitoring nurse responsive to comparing the dialysis treatment information to the patient log.

15. The method of claim 1, further comprising providing instructions to the patient based on the dialysis treatment information using the processor.

16. The method of claim 15, wherein the instructions are selected from the group consisting of instructions to change the dialysate, instructions to change the medication within the dialysate, instructions to change the concentration of the medication within the dialysate and instructions to change the dosage of the medication within the dialysate.

17. The method of claim 15, wherein the instructions are selected from group consisting of instructions to change a medication dosage, instructions to change a frequency at which a medication is taken, instructions to change a medication, instructions to contact a caregiver, instructions to provide additional dialysis treatment information.

18. The method of claim 17, wherein the information relating to the dialysate is selected from the group consisting of the type of dialysate, an identification of a medication within the dialysate, a concentration of the medication within the dialysate, a dosage of the medication within the dialysate.

19. The method of claim 1, wherein the dialysis treatment information further comprises fluid information selected from the group consisting of information relating to a dialysate used in the dialysis treatment, a volume or weight of administered dialysate, a volume or weight of the dialysate effluent recovered during the dialysis treatment and a concentration of glucose in the dialysate.

20. The method of claim 1, wherein the dialysis treatment information further comprises symptom information relating to an infection, an access issue, an access blockage, or other access matter requiring more immediate attention.

21. The method of claim 1, wherein dialysis treatment information further comprises:
symptom information relating to potential symptoms the patient may be experiencing in relation to the dialysis treatment selected from the group consisting of:
a color of a dialysate effluent, a cloudiness level of the dialysate effluent,
an assessment of an amount of swelling around a dialysis treatment access to the patient,
an assessment of a level of redness around the dialysis treatment access,
a soreness of the area around the dialysis treatment access,
a length of a catheter, a temperature of the patient,
a blood pressure of the patient, a weight of the patient before the dialysis treatment,
a weight of the patient after the dialysis treatment,
a photo of the dialysate effluent, and
a photo of the area around the dialysis treatment access.

22. A dialysis treatment monitoring system comprising:
an output device configured to present queries to a patient, wherein the queries are related to dialysis treatment of the patient;
an input device configured to receive patient responses to the queries;
a processor configured to collect and transmit the patient responses; and
a remote processing location configured to receive the patient responses and determine whether an alert should be sent to a healthcare professional based on the patient responses.

23. A dialysis treatment monitoring system, comprising:
at least one communication device configured to connect a patient to an interactive voice recognition system, wherein the interactive voice recognition system collects dialysis treatment information from the patient through a series of queries and responses,
wherein the dialysis treatment information is transmitted to a remote processing unit,
and wherein the remote processing unit processes the dialysis treatment information and determines whether an alert should be sent to a healthcare professional.
